(19) United States
(12) Patent Application Publication
(10) Pub. No.: US 2025/0235354 A1
(43) Pub. Date: Jul. 24, 2025

(54) MOBILE ROLL CURTAIN FOR A WELDING TABLE

(71) Applicant: Lincoln Global, Inc., Santa Fe Springs, CA (US)

(72) Inventor: William A. Reiser, Berea, OH (US)

(21) Appl. No.: 18/421,400

(22) Filed: Jan. 24, 2024

(51) Int. Cl. A61F 9/06

(52) U.S. Cl. CPC A61F 9/061

(57) ABSTRACT

An apparatus for blocking arc flash in an arc welding environment is provided. A roller assembly has a housing and a bottom portion with a first protruding peg element. The roller assembly is configured to be mounted at any of multiple discrete positions on a top portion of a welding table via the first protruding peg element. A handle assembly has a bottom end with a second protruding peg element. The handle assembly is configured to be mounted at any of the multiple discrete positions on the top portion of the welding table via the second protruding peg element. An arc shading curtain is mounted between an internal portion of the housing and the handle assembly. The roller assembly is configured to apply a rolling action to automatically retract the arc shading curtain into the housing. The apparatus is configured to be portable by a human user.

### FIELD

Embodiments of the present invention relate to welding manufacturing. More specifically, embodiments of the present invention relate to welding curtains used in arc welding manufacturing.

### BACKGROUND

In an arc welding manufacturing environment, persons in the welding environment who are not the welding operator can be exposed to the bright light (arc flash) of a welding arc if the light from the arc is not somehow shielded. Often, large welding curtains that stand on the floor of the welding environment are employed to shield the bright light from the arc created in a particular welding cell. However, such large welding curtains can take up a lot of floor space, are limited in how they can be set up, and can inadvertently act as obstacles to a person trying to get around within the welding environment.

### SUMMARY

Embodiments of apparatuses provide a mobile and modular welding roll curtain that is compact and can be placed strategically to prevent arc flash from manual or robotic welding applications. The mobile roll curtain is configured to be set up on a table-top of a welding table, providing set up flexibility such that the mobile roll curtain can be rapidly reconfigured on the welding table in various strategic positions and configurations. The mobile roll curtain is retractable, compact, and portable such that a user can easily travel from one welding environment to another with the mobile roll curtain. Also, with the mobile roll curtain, fume extraction from the welding work cell can be more efficient because large welding curtains are not being set up which can restrict air flow within the welding environment. One embodiment is an apparatus for blocking arc flash in an arc welding environment, having a roller assembly, a handle assembly, and an arc shading curtain as discussed below herein. One embodiment is a method of assembling an apparatus for blocking arc flash in an arc welding environment. The method includes assembling a roller assembly and a handle assembly, with an arc shading curtain mounted therebetween, as discussed below herein.

Numerous aspects of the general inventive concepts will become readily apparent from the following detailed description of exemplary embodiments, from the claims, and from the accompanying drawings.

### BRIEF DESCRIPTION OF THE DRAWINGS

The accompanying drawings, which are incorporated in and constitute a part of the specification, illustrate various embodiments of the disclosure. It will be appreciated that the illustrated element boundaries (e.g., boxes, groups of boxes, or other shapes) in the figures represent one embodiment of boundaries. In some embodiments, one element may be designed as multiple elements or that multiple elements may be designed as one element. In some embodiments, an element shown as an internal component of another element may be implemented as an external component and vice versa. Furthermore, elements may not be drawn to scale.

FIG. 1A illustrates a first view of one embodiment of an apparatus for blocking arc flash in an arc welding environment, with an arc shading curtain in an un-extended (rolled up) state;

FIG. 1B illustrates a second view of the apparatus of FIG. 1A in the un-extended state.

FIG. 2 illustrates the apparatus of FIG. 1A mounted on a tabletop in an arc welding environment and shown in the un-extended state;

FIG. 3 illustrates the apparatus of FIG. 1A with an arc shading curtain in an extended state, where the apparatus is mounted on a tabletop in an arc welding environment;

FIG. 4 illustrates the apparatus of FIG. 1A with an arc shading curtain in an angled and extended state, where the apparatus is mounted on a tabletop in an arc welding environment;

FIG. 5 illustrates the apparatus of FIG. 1A with the arc shading current in an angled and extended state, and where a portion of the apparatus is mounted on tooling that extends just off of the welding tabletop in an arc welding environment.

FIG. 6 illustrates one embodiment of a handle assembly and a roller assembly of an apparatus for blocking arc flash in an arc welding environment;

FIG. 7 illustrates one embodiment of a top cap element of the roller assembly of FIG. 6;

FIG. 8 illustrates one embodiment of a bottom cap element of the roller assembly of FIG. 6;

FIG. 9 illustrates one embodiment of a cup element of the roller assembly of FIG. 6;

FIG. 10 illustrates one embodiment of two unassembled halves of a rotatable inner shaft of the roller assembly of FIG. 6;

FIG. 11 illustrates one embodiment of two assembled halves of the handle assembly of FIG. 6;

FIG. 12 illustrates one embodiment of the peg element (uninserted) of the handle assembly of FIG. 6, or the peg element (uninserted) of the bottom cap element of FIG. 8;

FIG. 13 illustrates one embodiment of two assembled halves of the rotatable inner shaft of FIG. 10 having a first top cup element of FIG. 9 loaded with a first rotor spring and positioned on a top end of the rotatable inner shaft; and

FIG. 14 is a flowchart of one embodiment of a method of assembling an apparatus for blocking arc flash in an arc welding environment.

### DETAILED DESCRIPTION

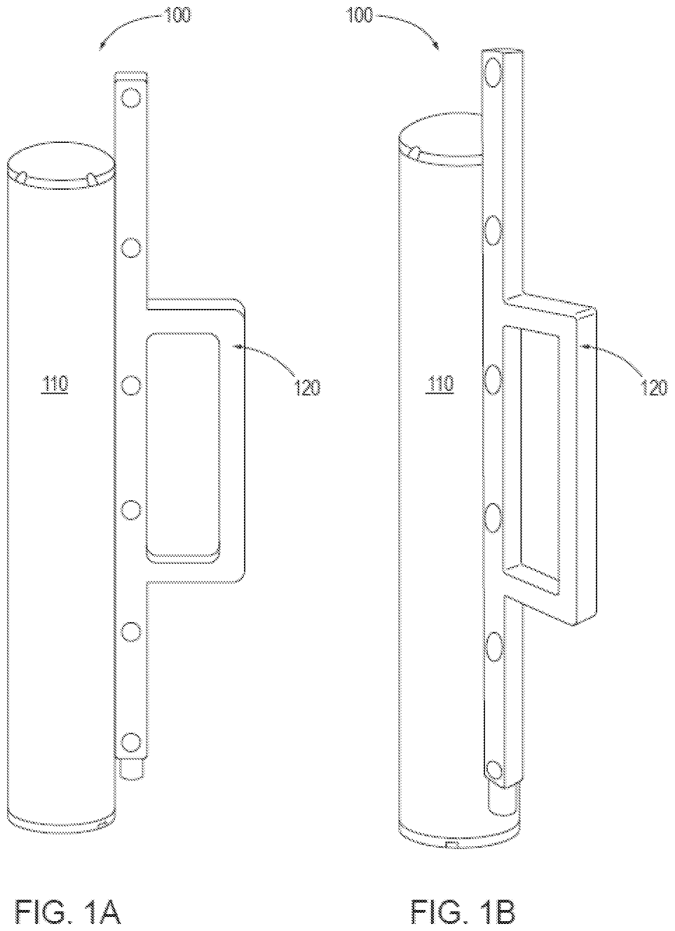

Embodiments of the present invention include systems and methods to block arc flash in an arc welding environment. The examples and figures herein are illustrative only and are not meant to limit the subject invention, which is measured by the scope and spirit of the claims. Referring now to the drawings, wherein the showings are for the purpose of illustrating exemplary embodiments of the subject invention only and not for the purpose of limiting same, FIG. 1A illustrates a first view of one embodiment of an apparatus 100 for blocking arc flash in an arc welding environment, with an arc shading curtain in an un-extended (rolled up) state. FIG. 1B illustrates a second view of the apparatus 100 of FIG. 1A in the un-extended state. As seen in FIG. 1A and FIG. 1B, the apparatus 100 has a roller assembly 110 and a handle assembly 120. Upon a user pulling the handle assembly 120, an arc shading curtain is rolled out of (extended from) a slit in a housing of the roller assembly 110.

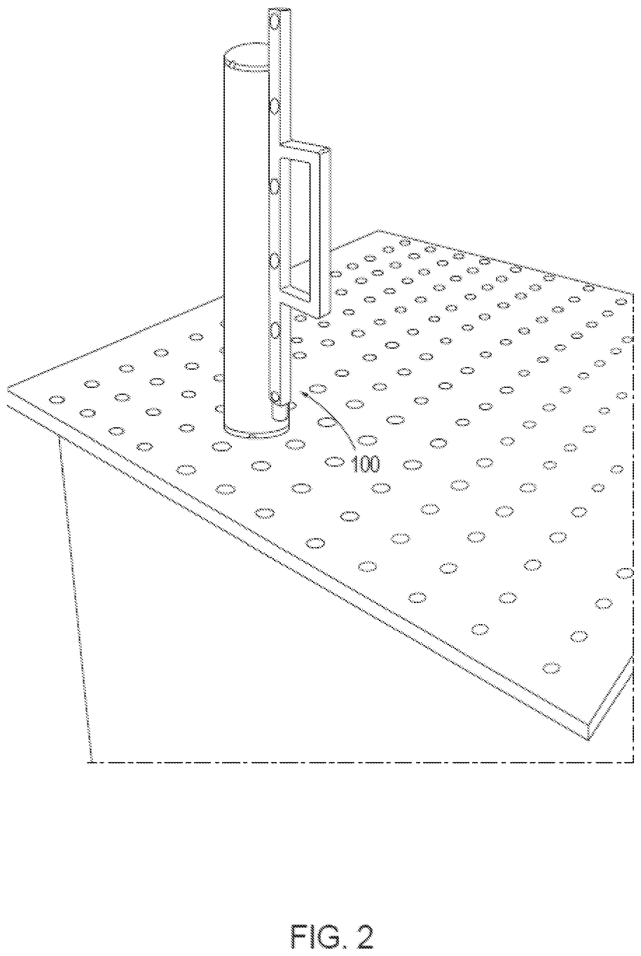

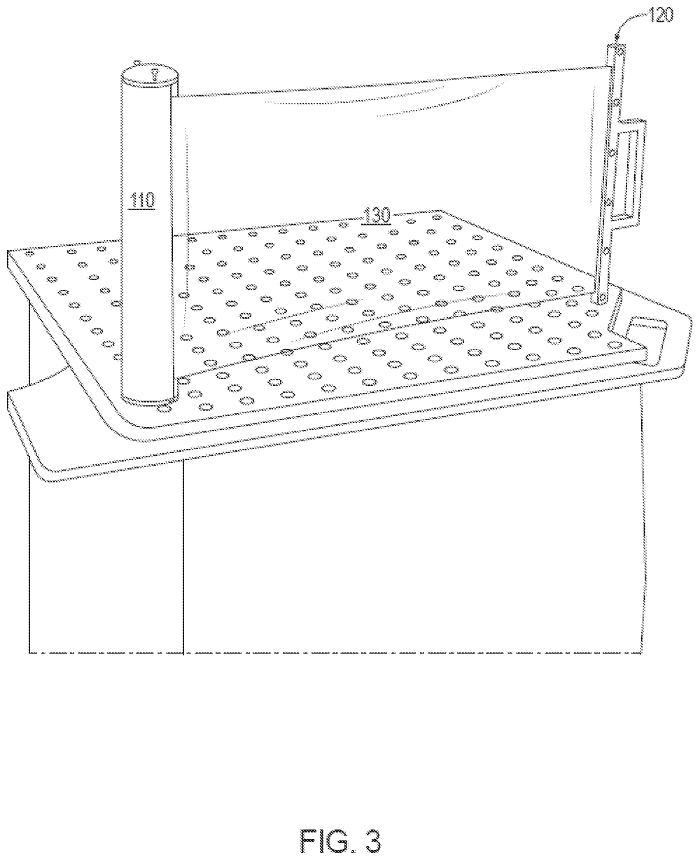

FIG. 2 illustrates the apparatus 100 of FIG. 1A mounted on a welding tabletop in an arc welding environment and shown in the un-extended state (i.e., the arc shading curtain is rolled up inside the housing of the roller assembly 110). FIGS. 3-5 illustrate that the apparatus 100 can be set up in any of multiple different configurations on, or just off of, the welding tabletop. For example, FIG. 3 illustrates the apparatus 100 of FIG. 1A with an arc shading curtain 130 in an extended state (i.e., the arc shading curtain 130 is pulled out of the housing of the roller assembly 100), where the apparatus 100 is mounted on a welding tabletop having multiple mounting holes in an arc welding environment. The bottom portion of the roller assembly 110 and the bottom portion of the handle assembly 120 have protruding peg elements (discussed later herein) for mounting the apparatus 100 into the mounting holes of the welding tabletop.

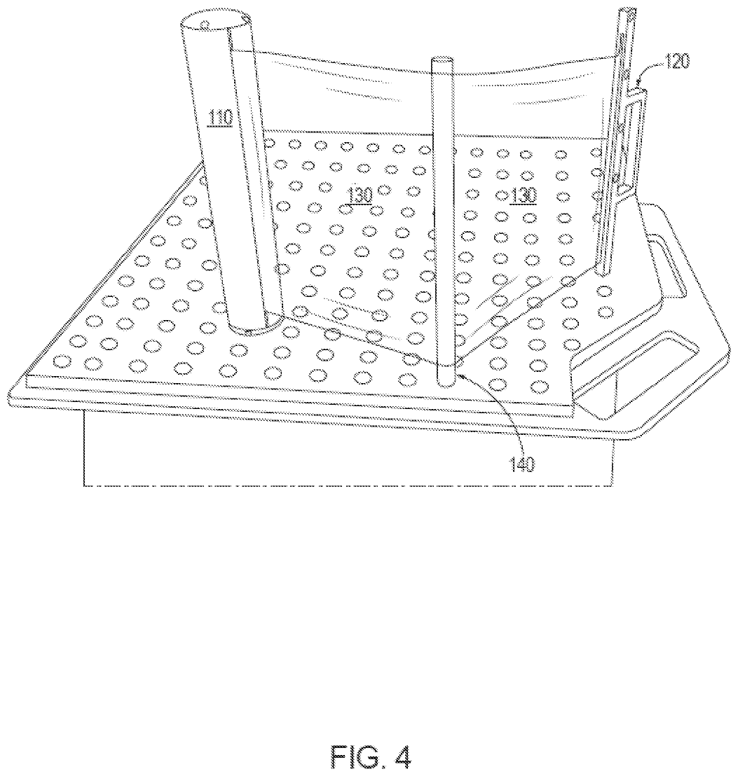

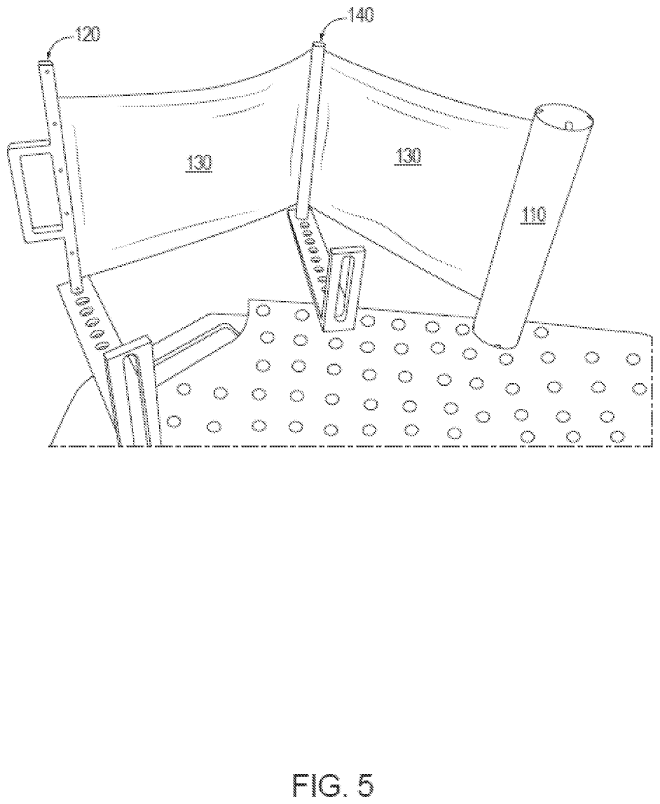

FIG. 4 illustrates the apparatus 100 of FIG. 1A with the arc shading curtain 130 in an angled and extended state, where the apparatus 100 is mounted on the welding tabletop in an arc welding environment. FIG. 5 illustrates the apparatus 100 of FIG. 1A with the arc shading current 130 in an angled and extended state, and where a portion of the apparatus 100 is mounted on tooling that extends just off of the welding tabletop in an arc welding environment. The angled and extended states use a curtain post 140 to effectively allow the arc shading curtain to be angled around the curtain post 140. It will be apparent to one skilled in the art that other set up configurations are possible as well, due to the multiple discrete holes in the welding tabletop and other possible tooling that could be employed.

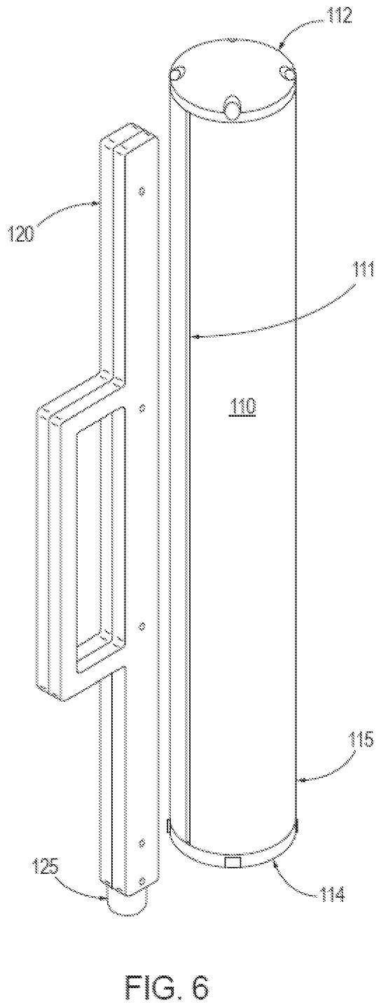

FIG. 6 illustrates one embodiment of the handle assembly 120 and the roller assembly 110 of the apparatus 100 for blocking arc flash in an arc welding environment. The handle assembly 120 has a bottom end with a protruding peg element 125. The handle assembly is configured to be mounted at any of the multiple discrete positions on the top portion of the welding table via the protruding peg element 125. The protruding peg element 125 is interchangeable with at least one other peg element such that the handle assembly 120 can be compatible with at least two different types of welding tables having different hole sizes and/or hole shapes at any of the multiple discrete positions on top portions of the respective welding tables. The roller assembly 110 of the apparatus 100 has a housing 115 with a slit 111 and is configured to stop a rolling action during retraction of the arc shading curtain 130 through the slit 111 when the handle assembly 120 meets the housing 115 and the arc shading curtain is fully retracted into the housing 115.

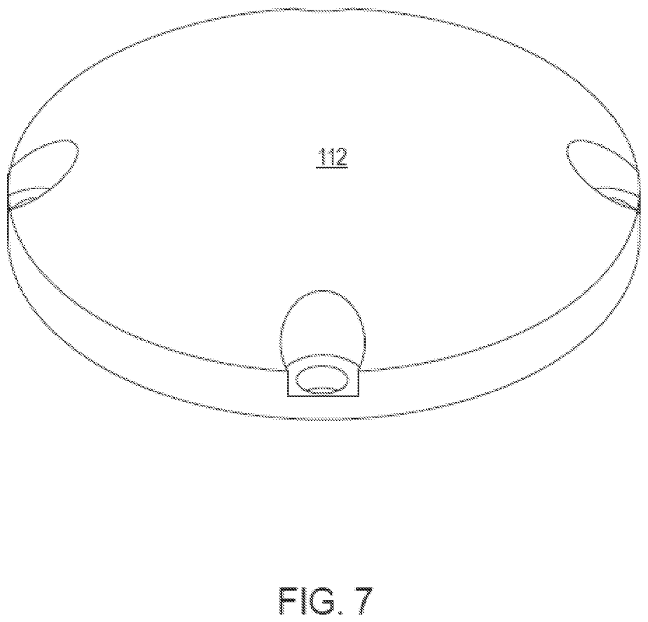

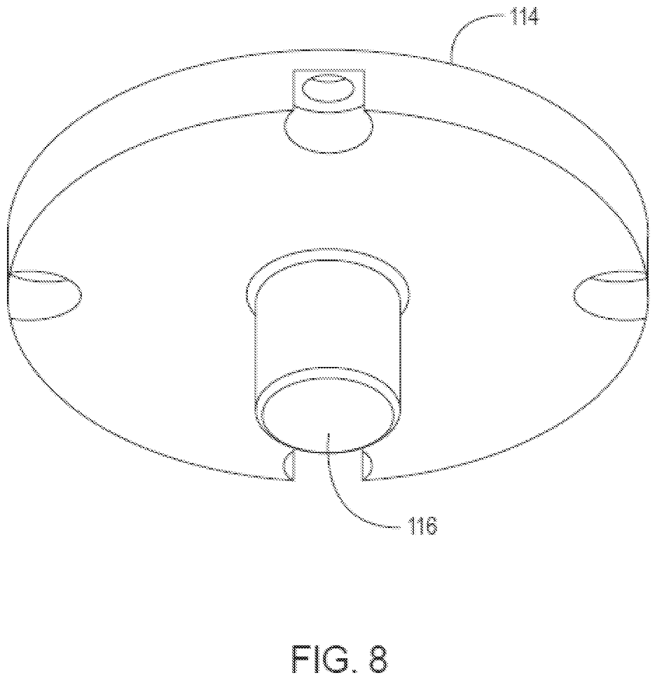

FIG. 6 also shows a top cap element 112 and a bottom cap element 114 of the roller assembly 110. FIG. 7 illustrates one embodiment of the top cap element 112 of the roller assembly 110 of FIG. 6. FIG. 8 illustrates one embodiment of the bottom cap element 114 of the roller assembly 110 of FIG. 6. The bottom cap element 114 is configured to accept a protruding peg element 116 that is interchangeable with at least one other peg element such that the roller assembly 110 can be compatible with at least two different types of welding tables having different hole sizes and/or hole shapes at any of multiple discrete positions on top portions of the respective welding tables. The top cap element 112 may be mounted at the top end of the housing 115 and the bottom cap element 114 may be mounted at the bottom end of the housing 115 via at least one of screws, bolts, clamps, snapping type connectors, hinges, a compression fitting, and a threaded fitting.

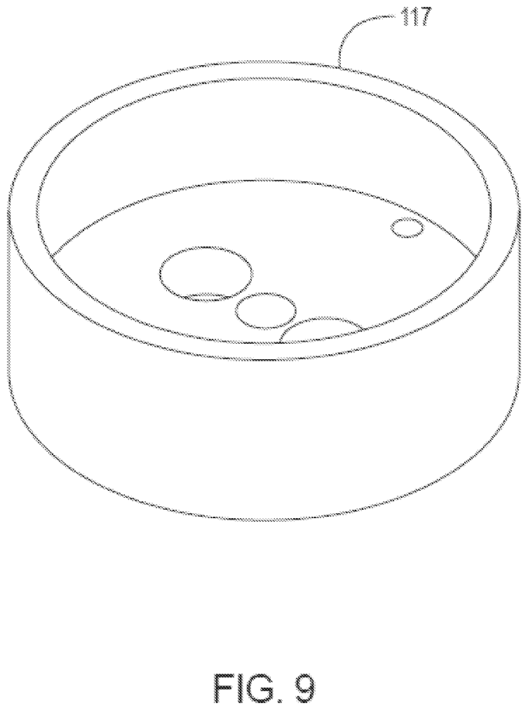

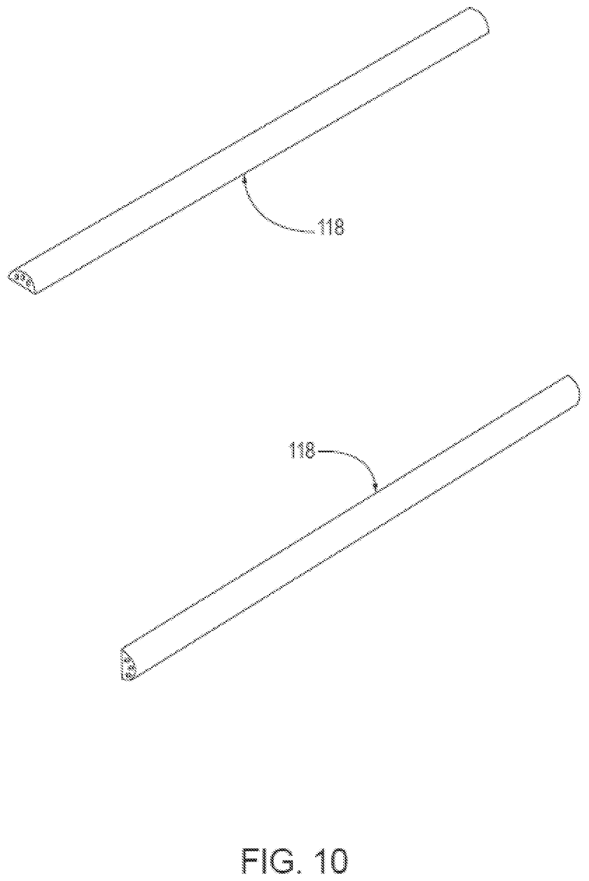

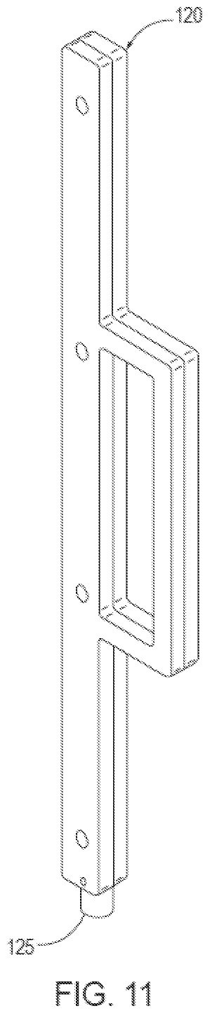

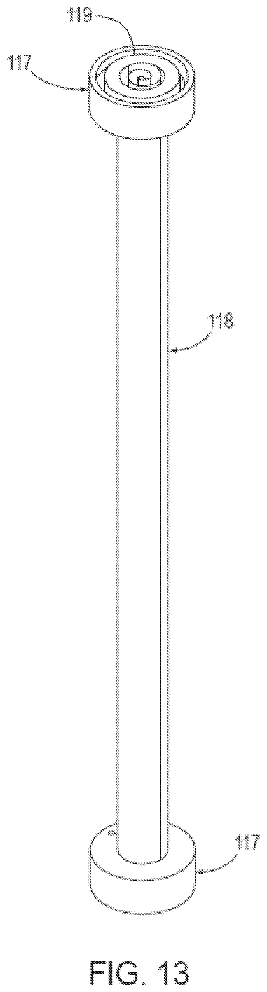

FIG. 9 illustrates one embodiment of a cup element 117 of the roller assembly 110 of FIG. 6. Two cup elements 117 are used in the roller assembly 110, and each cup element 117 contains a rotor spring 119 (as shown in and discussed with respect to FIG. 13). FIG. 10 illustrates one embodiment of two unassembled halves of a rotatable inner shaft 118 of the roller assembly 110 of FIG. 6. The two halves of the rotatable inner shaft 118 are assembled by having one side of the arc shading curtain 130 sandwiched between the two halves. FIG. 11 illustrates one embodiment of two assembled halves of the handle assembly 120 of FIG. 6. The two halves of the handle assembly 120 are assembled by having the other side of the arc shading curtain 130 sandwiched between the two halves. The peg element 125 is shown at a bottom end of the handle assembly 120.

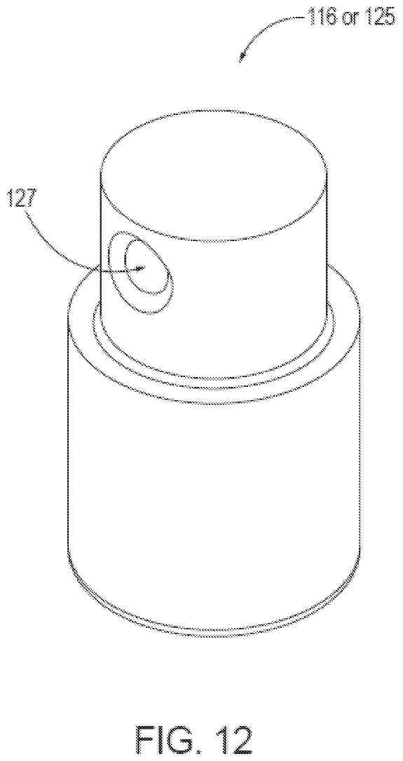

FIG. 12 illustrates one embodiment of the peg element 125 (uninserted) of the handle assembly 120 of FIG. 6, or the peg element 116 (uninserted) of the bottom cap element 114 of FIG. 8. Again, the protruding peg element 125 is interchangeable with at least one other peg element (of a different size and/or shape) such that the handle assembly 120 can be compatible with at least two different types of welding tables having different hole sizes and/or hole shapes at any of the multiple discrete positions on top portions of the respective welding tables. Similarly, the protruding peg element 116 is interchangeable with at least one other peg element (of a different size and/or shape) such that the roller assembly 110 can be compatible with at least two different types of welding tables having different hole sizes and/or hole shapes at any of multiple discrete positions on top portions of the respective welding tables. In one embodiment, each peg element has a locking hole 127 that facilitates locking of the peg element into the bottom cap element 114 of the roller assembly 110, the bottom end of the handle assembly 120, or the bottom end of the curtain post 140.

FIG. 13 illustrates one embodiment of two assembled halves of the rotatable inner shaft 118 of FIG. 10 having a first top cup element 117 of FIG. 9 loaded with a first rotor spring 119 and positioned on a top end of the rotatable inner shaft 118. Furthermore, a second bottom cup element 117, with a second rotor spring 119, is positioned on a bottom end of the assembled rotatable inner shaft 118. The respective cup elements 117 and rotor springs 119 are configured and positioned such that the first rotor spring 119 and the second rotor spring 119 can interact with the rotatable inner shaft 118 to retract the arc shading curtain 130. Essentially, as the user pulls the handle assembly 120, the springs 119 coil around themselves, inside the cups 117, and the resulting tension pulls the curtain 130 back, to self-rewind around the inner shaft 118, once the user stops pulling. The inner shaft 118 and cups 117 rotate, as the arc shading curtain 130 is pulled via the handle assembly 120, inside the housing 115. The caps 117 on the top and bottom have a pin that keep the end of the corresponding spring 119, located in the center of the coiled spring 119, stationary.

In summary, one embodiment is an apparatus 100 for blocking arc flash in an arc welding environment. A roller assembly 110 of the apparatus 100 has a housing 115 and a bottom portion 114 with a first protruding peg element 116. The roller assembly 110 is configured to be mounted at any of multiple discrete positions on a top portion of a welding table via the first protruding peg element 116. A handle assembly 120 of the apparatus 100 has a bottom end with a second protruding peg element 125. The handle assembly 120 is configured to be mounted at any of the multiple discrete positions on the top portion of the welding table via the second protruding peg element 125. An arc shading curtain 130 of the apparatus is mounted between an internal portion of the housing 115 and the handle assembly 120. The roller assembly 110 is configured to apply a rolling action to automatically retract the arc shading curtain 130 into the housing 115. The apparatus 100 is configured to be easily portable by a human user.

In one embodiment, the apparatus 100 is configured to stop the rolling action by the handle assembly 120 meeting the housing 115 when the arc shading curtain 130 is fully retracted. The first protruding peg element 116 is interchangeable with at least one other peg element such that the roller assembly 110 can be compatible with at least two different types of welding tables having different hole sizes and/or hole shapes at any of the multiple discrete positions on top portions of the respective welding tables. The second protruding peg element 125 is interchangeable with at least one other peg element such that the handle assembly 120 can be compatible with at least two different types of welding tables having different hole sizes and/or hole shapes at any of the multiple discrete positions on top portions of the respective welding tables.

In one embodiment, the apparatus 100 includes at least one curtain post 140 having a third protruding peg element (not shown, but similar to the first and second protruding peg elements). The curtain post 140 is configured to be mounted at any of the multiple discrete positions on the top portion of the welding table via the third protruding peg element. In one embodiment, the housing 115 is made of at least one of aluminum, an aluminum alloy, or a plastic. In one embodiment, the handle assembly 120 is made of at least one of aluminum, an aluminum alloy, or a plastic. In one embodiment, the roller assembly 110 includes a rotatable inner shaft 118 located within the internal portion of the housing 115 and having a first side of the arc shading curtain 130 mounted thereto. In one embodiment, the arc shading curtain 130 is made of a flexible material that is configured to mitigate arc flash through the arc shading curtain. In one embodiment, the roller assembly 110 includes at least two rotor spring elements 119 to provide the rolling action.

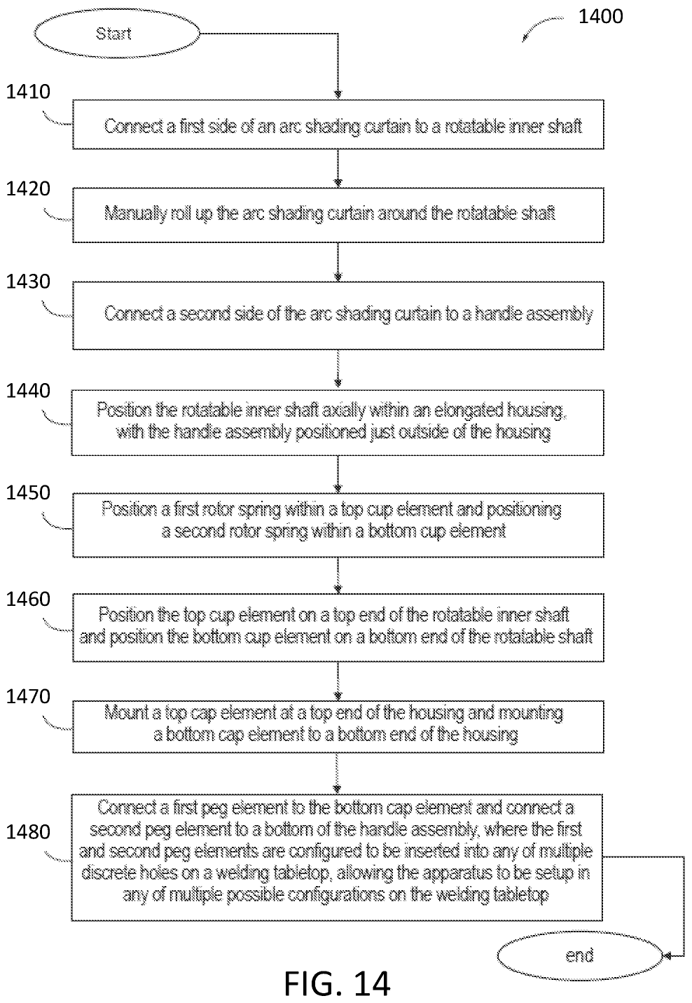

FIG. 14 is a flowchart of one embodiment of a method 1400 of assembling an apparatus 100 for blocking arc flash in an arc welding environment. Referring to FIG. 14, at block 1410 of FIG. 14, the method 1400 includes connecting a first side of an arc shading curtain 130 to a rotatable inner shaft 118. At block 1420, the method 1400 includes manually rolling up the arc shading curtain 130 around the rotatable inner shaft 118. At block 1430, the method 1400 includes connecting a second side of the arc shading curtain 130 to a handle assembly 120. At block 1440, the method 1400 includes positioning the rotatable inner shaft 118, with the arc shading curtain 130 as rolled up, axially within an elongated housing 115, with the handle assembly 120 positioned just outside of the housing 115 as facilitated by a slot 111 running along a length of the housing 115 to accommodate the arc shading curtain 130 therethrough.

At block 1450, the method 1400 includes positioning a first rotor spring 119 within a top cup element 117 and positioning a second rotor spring 119 within a bottom cup element 117. At block 1460, the method 1400 includes positioning the top cup element 117, with the first rotor spring 119, on a top end of the rotatable inner shaft 118 and positioning the bottom cup element 117, with the second rotor spring 119, on a bottom end of the rotatable inner shaft 118 such that the first rotor spring 119 and the second rotor spring 119 can interact with the rotatable inner shaft 118 to retract the arc shading curtain 130.

At block 1470, the method 1400 includes mounting a top cap element 112 at a top end of the housing 115 and mounting a bottom cap element 114 at a bottom end of the housing 115. At block 1480, the method 1400 includes connecting a first peg element 116 to the bottom cap element 114 and connecting a second peg element 125 to a bottom end of the handle assembly 120. The first peg element 116 and the second peg element 125 are each configured to be inserted into any of multiple discrete holes on a top portion of a welding table, allowing the apparatus 100 to be set up in any of multiple possible configurations on the top portion of the welding table with the arc shading curtain partially extended or fully extended from the housing 115.

A rolling action, provided by the first and second rotor springs 119 acting upon the rotatable inner shaft 118 during retraction of the arc shading curtain 130, is stopped when the handle assembly 120 meets the housing 115. The first peg element 116 is interchangeable with at least one other peg element such that the roller assembly 110 can be compatible with at least two different types of welding tables having different hole sizes/shapes at any of the multiple discrete holes on top portions of the respective welding tables. Similarly, the second peg element 125 is interchangeable with at least one other peg element such that the handle assembly 120 can be compatible with at least two different types of welding tables having different hole sizes/shapes at any of the multiple discrete holes on top portions of the respective welding tables. In one embodiment, the curtain post 140 is configured to be employed by a user to set up the arc shading curtain 130 in any of multiple possible angled configurations on a top portion of a welding table with the arc shading curtain 130 partially extended or fully extended from the housing 115 via pulling of the handle assembly 120.

In one embodiment, connecting the first side of the arc shading curtain 130 to the rotatable inner shaft 118 includes sandwiching the first side between two halves of the rotatable inner shaft 118. Similarly, connecting the second side of the arc shading curtain 130 to the handle assembly 120 includes sandwiching the second side between two halves of the handle assembly 120. Mounting the top cap element 112 at the top end of the housing 115 and mounting the bottom cap element 114 to the bottom end of the housing 115 are accomplished by at least one of screws, bolts, clamps, snapping type connectors, hinges, a compression fitting, or a threaded fitting, in accordance with various embodiments.

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or illustrative examples shown and described. Thus, this disclosure is intended to embrace alterations, modifications, and variations that fall within the scope of the appended claims, which satisfy the statutory subject matter requirements of 35 U.S.C. § 101. The above description of specific embodiments has been given by way of example. From the disclosure given, those skilled in the art will not only understand the general inventive concepts and attendant advantages, but will also find apparent various changes and modifications to the structures and methods disclosed. It is sought, therefore, to cover all such changes and modifications as fall within the spirit and scope of the general inventive concepts, as defined by the appended claims, and equivalents thereof.

What is claimed is:

1. An apparatus for blocking arc flash in an arc welding environment, the apparatus comprising:
a roller assembly having a housing and a bottom portion with a first protruding peg element, wherein the roller assembly is configured to be mounted at any of multiple discrete positions on a top portion of a welding table via the first protruding peg element;
a handle assembly having a bottom end with a second protruding peg element, wherein the handle assembly is configured to be mounted at any of the multiple discrete positions on the top portion of the welding table via the second protruding peg element; and
an arc shading curtain mounted between an internal portion of the housing and the handle assembly,
wherein the roller assembly is configured to apply a rolling action to automatically retract the arc shading curtain into the housing, and wherein the apparatus is configured to be portable by a human user.

2. The apparatus of claim 1, wherein the apparatus is configured to stop the rolling action by the handle assembly meeting the housing when the arc shading curtain is fully retracted.

3. The apparatus of claim 1, wherein the first protruding peg element is interchangeable with at least one other peg element such that the roller assembly can be compatible with at least two different types of welding tables having different hole sizes at any of the multiple discrete positions on top portions of the respective welding tables.

4. The apparatus of claim 1, wherein the second protruding peg element is interchangeable with at least one other peg element such that the handle assembly can be compatible with at least two different types of welding tables having different hole sizes at any of the multiple discrete positions on top portions of the respective welding tables.

5. The apparatus of claim 1, further comprising at least one curtain post having a third protruding peg element, wherein the at least one curtain post is configured to be mounted at any of the multiple discrete positions on the top portion of the welding table via the third protruding peg element.

6. The apparatus of claim 1, wherein the housing is made of at least one of aluminum, an aluminum alloy, or a plastic.

7. The apparatus of claim 1, wherein the handle assembly is made of at least one of aluminum, an aluminum alloy, or a plastic.

8. The apparatus of claim 1, wherein the roller assembly further includes a rotatable inner shaft located within the internal portion of the housing and has a first side of the arc shading curtain mounted thereto.

9. The apparatus of claim 1, wherein the arc shading curtain is made of a flexible material that is configured to mitigate arc flash through the arc shading curtain.

10. The apparatus of claim 1, wherein the roller assembly further includes at least two rotor spring elements to provide the rolling action.

11. A method of assembling an apparatus for blocking arc flash in an arc welding environment, the method comprising:
connecting a first side of an arc shading curtain to a rotatable inner shaft;
manually rolling up the arc shading curtain around the rotatable inner shaft;
connecting a second side of the arc shading curtain to a handle assembly;
positioning the rotatable inner shaft, with the arc shading curtain as rolled up, axially within an elongated housing, with the handle assembly positioned just outside of the housing as facilitated by a slot running along a length of the housing to accommodate the arc shading curtain therethrough;
positioning a first rotor spring within a top cup element and positioning a second rotor spring within a bottom cup element;
positioning the top cup element, with the first rotor spring, on a top end of the rotatable inner shaft and positioning the bottom cup element, with the second rotor spring, on a bottom end of the rotatable shaft such that the first rotor spring and the second rotor spring can interact with the rotatable inner shaft to retract the arc shading curtain; and
configuring a curtain post to be employed by a user to set up the arc shading curtain in any of multiple possible angled configurations on a top portion of a welding table with the arc shading curtain partially extended or fully extended from the housing via pulling of the handle assembly.

12. The method of claim 1, wherein a rolling action, provided by the first rotor spring and the second rotor spring acting upon the rotatable inner shaft during retraction of the arc shading curtain, is stopped when the handle assembly meets the housing.

13. The method of claim 1, wherein connecting the first side of the arc shading curtain to the rotatable inner shaft includes sandwiching the first side between two halves of the rotatable inner shaft.

14. The method of claim 1, wherein connecting the second side of the arc shading curtain to the handle assembly includes sandwiching the second side between two halves of the handle assembly.

15. The method of claim 1, further including:
mounting a top cap element at a top end of the housing and mounting a bottom cap element to a bottom end of the housing; and
connecting a first peg element to the bottom cap element and connecting a second peg element to a bottom end of the handle assembly, wherein the first peg element and the second peg element are each configured to be inserted into any of multiple discrete holes on a top portion of a welding table, allowing the apparatus to be set up in any of multiple possible configurations on the top portion of the welding table with the arc shading curtain partially extended or fully extended from the housing.

16. The method of claim 15, wherein the first peg element is interchangeable with at least one other peg element such that the apparatus can be compatible with at least two different types of welding tables having different hole sizes at any of the multiple discrete holes on top portions of the respective welding tables.

17. The method of claim 15, wherein the second peg element is interchangeable with at least one other peg element such that the handle assembly can be compatible with at least two different types of welding tables having different hole sizes at any of the multiple discrete holes on top portions of the respective welding tables.

18. The method of claim 15, wherein the mounting of the top cap element at the top end of the housing and the mounting of the bottom cap element to the bottom end of the housing are accomplished by at least one of screws, bolts, clamps, snapping type connectors, hinges, a compression fitting, and a threaded fitting.

19. An apparatus for blocking arc flash in an arc welding environment, the apparatus comprising:
a roller assembly having a housing and a bottom portion with a first protruding peg element;
a handle assembly having a bottom end with a second protruding peg element;
an arc shading curtain mounted between an internal portion of the housing and the handle assembly; and
a curtain post configured to be employed by a user to set up the arc shading curtain in any of multiple possible angled configurations on a top portion of a welding table with the arc shading curtain partially extended or fully extended from the housing via pulling of the handle assembly,
wherein the roller assembly is configured to apply a rolling action to automatically retract the arc shading curtain into the housing, and wherein the apparatus is configured to be portable by a human user.

20. The apparatus of claim 19, wherein the roller assembly is configured to be mounted at any of multiple discrete positions on the top portion of the welding table via the first protruding peg element, and wherein the handle assembly is configured to be mounted at any of the multiple discrete positions on the top portion of the welding table via the second protruding peg element.
